(19) United States
(12) Patent Application Publication
(10) Pub. No.: US 2025/0006350 A1
(43) Pub. Date: Jan. 2, 2025

(54) CALLING SYSTEM

(71) Applicant: SINTOKOGIO, LTD., Nagoya-shi (JP)

(72) Inventors: Yoshikane TANAAMI, Nagoya-shi (JP); Miyuki HAYASHI, Nagoya-shi (JP)

(73) Assignee: SINTOKOGIO, LTD., Nagoya-shi (JP)

(21) Appl. No.: 18/740,723

(22) Filed: Jun. 12, 2024

(30) Foreign Application Priority Data
Jun. 30, 2023 (JP) 2023-108646

(51) Int. Cl. G16H 40/20

(52) U.S. Cl. CPC G16H 40/20

(57) ABSTRACT

A calling system includes: a mutual capacitive contact sensor which has fibrous electric conductors, the mutual capacitive contact sensor being formable integrally with a garment to be worn by a first person or being attachable to the garment; a first information provision section configured to provide the first person with first information for assisting the first person in operating the contact sensor; a second information provision section configured to provide a second person with second information for calling the second person; a control section configured to cause the second information provision section to provide the second information on the basis of an operation by the first person for the contact sensor, so as to execute a calling process of calling the second person.

This Nonprovisional application claims priority under 35 U.S.C. § 119 on Patent Application No. 2023-108646 filed in Japan on Jun. 30, 2023, the entire contents of which are hereby incorporated by reference.

### TECHNICAL FIELD

The present invention relates to a calling system that allows a first person to call a second person.

### BACKGROUND ART

Patent Literature 1 discloses a technology of a sensor system that monitors a body surface with use of a fabric-based pressure sensor. This sensor system can provide conditions of the body surface to a user, a manager, and/or a clinician (see ABSTRACT of Patent Literature 1).

### CITATION LIST

### [Patent Literature]

### [Patent Literature 1]

Published Japanese Translation of PCT International Application Tokuhyo No. 2015-509028

### SUMMARY OF INVENTION

### Technical Problem

However, such a conventional technology merely provides conditions of a body surface to a clinician and/or the like and does not enable the user to call the clinician and/or the like.

It is an object of an aspect of the present invention to achieve a calling system that enables a first person to call a second person by operating a contact sensor disposed on a garment.

### Solution to Problem

In order to solve the foregoing problem, a calling system in accordance with an aspect of the present invention is a calling system that allows a first person to call a second person, the calling system including: a mutual capacitive contact sensor which has fibrous electric conductors and which is to be operated by the first person, the mutual capacitive contact sensor being formable integrally with a garment to be worn by the first person or being attachable to the garment; a first information provision section configured to provide the first person with first information for assisting the first person in operating the mutual capacitive contact sensor; a second information provision section configured to provide the second person with second information for calling the second person; and a control section configured to cause the second information provision section to provide the second information in accordance with an operation by the first person for the mutual capacitive contact sensor, so as to execute a calling process of calling the second person.

### Advantageous Effects of Invention

According to an aspect of the present invention, it is possible to achieve a calling system that enables a first person to call a second person by operating a contact sensor disposed on a garment.

### BRIEF DESCRIPTION OF DRAWINGS

FIG. 1 is a view schematically illustrating a calling system in accordance with an embodiment of the present invention.

FIG. 2 is a flowchart illustrating a calling method in accordance with an embodiment of the present invention.

FIG. 3 is a view schematically illustrating a relationship between first information provided by a first information provision section and an operation of a contact sensor.

FIG. 4 is a view schematically illustrating an example of information on options for the calling reason which is provided by the first information provision section.

FIG. 5 is a view schematically illustrating an example of information on options provided by the first information provision section.

FIG. 6 is a view schematically illustrating an example of information on options provided by the first information provision section.

FIG. 7 is a view schematically illustrating an example of information on options provided by a second information provision section.

### DESCRIPTION OF EMBODIMENTS

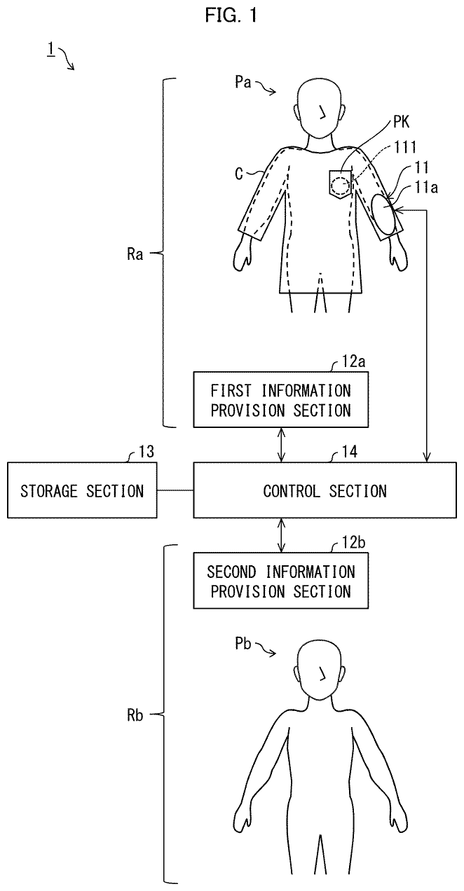

The following description will discuss an embodiment of the present invention in detail. FIG. 1 is a view schematically illustrating a calling system 1 in accordance with an embodiment of the present invention.

The calling system 1 is a system that allows a first person Pa to call a second person Pb. The first person Pa is, for example, a subject or operator of a medical/nursing care, and the second person Pb is, for example, a provider or supervisor of a medical/nursing care or a supervisor of an operation. It is assumed that the first person Pa and the second person Pb are present respectively in a region Ra and a region Rb which are spaced from each other.

The number of at least one of the first person Pa and the second person Pb may be two or more. As described later, a plurality of first persons Pa can call a second person Pb. Alternatively, a first person Pa can call a plurality of second persons Pb. Still alternatively, a plurality of first persons Pa and a plurality of second persons Pb may be present. In a case where, as described above, the number of at least one of the first person Pa and the second person Pb is two or more, the numbers of a first information provision section 12a and a second information provision section 12b, which will be described in the following section, preferably correspond respectively to the numbers of the first person Pa and the second person Pb.

The calling system 1 includes a contact sensor 11, a first information provision section 12a, a second information provision section 12b, a storage section 13, and a control section 14.

The contact sensor 11 is to be operated by the first person Pa and has a contact surface 11a with which the first person Pa can make a direct or indirect contact. By making a direct or indirect contact with the contact surface 11a, the first person Pa can operate the contact sensor 11 to call the second person Pb via the calling system 1. The first person Pa can operate the contact sensor 11, for example, by making a direct contact with the contact surface 11a with a fingertip. The first person Pa may also make an indirect contact with the contact surface 11a via an item (for example, a rod-like member) to operate the contact sensor 11. When the first person Pa makes contact with the contact surface 11a, a capacitance at a point on the contact surface 11a with which the first person Pa has made contact is changed. The contact sensor 11 can detect the change in a capacitance and thus the contact area.

The contact sensor 11 is a mutual capacitive contact sensor having fibrous electric conductors and can be formed integrally with a garment C. For example, a first fibrous electric conductor for generating an electric field and a second fibrous electric conductor for absorbing an electric field are woven into the garment to be worn by the first person Pa (for example, through needlework) in a lattice pattern, so that the contact sensor 11 can be formed integrally with the garment C. Alternatively, the contact sensor 11 may be configured to be attachable to the garment C. That is, for example, the contact sensor 11 may be configured in a form of a patch, a seal, or a badge and be attached to the garment C.

The contact sensor 11 may be formed integrally with the garment C to be worn by the first person Pa, in particular, the left sleeve and/or the right sleeve or may be attached to the left sleeve and/or the right sleeve of the garment C. In a case where the first person Pa is right-handed, the contact sensor 11 is disposed on the left sleeve of the garment C. In a case where the first person Pa is left-handed, the contact sensor 11 is disposed on the right sleeve of the garment C. This enables the first person Pa to operate the contact sensor 11 with a dominant hand. A pair of the contact sensors 11 may be attached to the respective left and right sleeves of the garment C. This configuration allows both a right-handed first person Pa and a left-handed first person Pa to easily operate the contact sensor 11 with the dominant hand.

The contact sensor 11 can have an auxiliary device 111. The auxiliary device 111 can include, for example, a voltage generation section for applying a voltage to an electrode of the contact sensor 11, a capacitive detection section for detecting a capacitance of the contact sensor 11, and a communication section for communicating with the control section 14. Communication between the communication section and the control section 14 may be performed in either a wired or wireless manner. The auxiliary device 111 can be disposed, for example, in a pocket PK of the garment C. Here, the auxiliary device 111 is separated from the contact sensor 11, but the auxiliary device 111 and the contact sensor 11 are integrated with each other.

The first information provision section 12a provides the first person Pa with first information that assists the first person Pa in operating the contact sensor 11. The second information provision section 12b provides the second person Pb with second information for calling the second person Pb. The first information provision section 12a and the second information provision section 12b are each connected with the control section 14 in a wired or wireless manner and each provide the information on the basis of an instruction from the control section 14.

Each of the first information provision section 12a and the second information provision section 12b can be constituted by, for example, an image display device, a sound output device, or a combination thereof. Examples of the image display device include a liquid crystal display device and an electro luminescence (EL) display device. Examples of the sound output device include speakers and earphones. In this case, the first information provision section 12a and the second information provision section 12b provide first information and second information respectively, as image information, sound information, and/or the like.

The first information provision section 12a and the second information provision section 12b are preferably disposed in the region Ra in which the first person Pa is present and the region Rb in which the second person Pb is present, respectively. Note that the storage section 13 and the control section 14 can be disposed as appropriate in the region Ra, the region Rb, and other regions.

The storage section 13 is constituted by a hardware resource for storing information, for example, a secondary storage device (for example, a flash memory, a hard disk, or a solid state drive (SSD)) and stores a table T indicative of correspondences between a plurality of positions on the contact surface 11a and a plurality of options. The table T is, for example, information indicative of correspondences between ranges of coordinates on the contact surface 11a and options corresponding to the ranges.

The control section 14 is constituted by, for example, a processor (for example, a central processing unit (CPU)). Note that as the control section 14 and the storage section 13, computers, e.g., personal computers (PCs) or server devices may be used.

The control section 14 executes a calling process of calling the second person Pb by causing the second information provision section 12b to provide the second information on the basis of an operation by the first person Pa for the contact sensor 11. The control section 14 can accept an option selected by the first person Pa, on the basis of the table T and a position on the contact surface 11a with which the first person Pa has made contact, so as to execute a calling method S10 described later.

The control section 14 can control the first information provision section 12a to cause the first information provision section 12a to provide information indicative of correspondence relations between the plurality of positions on the contact surface 11a and a plurality of options, as the first information. Examples of the first information include an image in which a plurality of options are disposed. Data on this image may be stored in the storage section 13 in advance or may be generated by the control section 14 on the basis of the table T. Provision of the first information enables the first person Pa to easily select an option with use of the contact sensor 11.

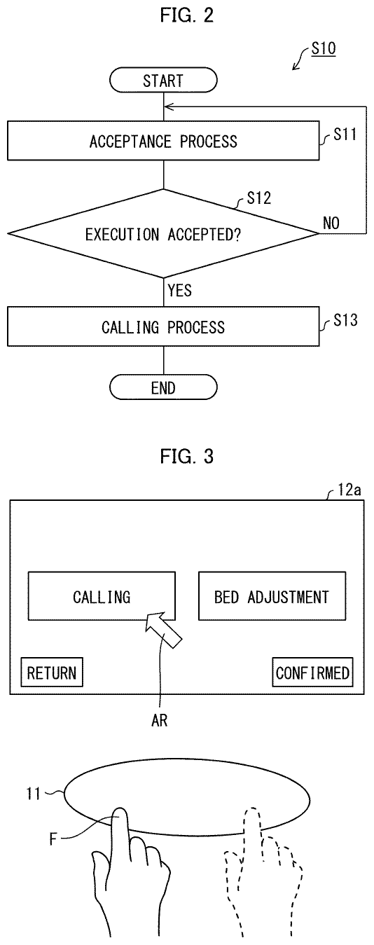

FIG. 2 is a flowchart illustrating a calling method S10 in accordance with an embodiment of the present invention. The calling method S10 executes acceptance processes S11 and a calling process S13. The following description will discuss the calling method S10 in detail.

The control section 14 executes the plurality of acceptance processes S11. The first person Pa operates the contact sensor 11 to select an option. In this case, the control section 14 controls the first information provision section 12a to cause the first information provision section 12a to provide the first information, so as to assist the first person Pa in selecting an option. The control section 14 accepts the option selected by the first person Pa on the basis of the table T and a position on the contact surface 11a with which the first person Pa has made contact.

The acceptance processes S11 are, for example, a first acceptance process, a second acceptance process, and a third acceptance process which will be described below. The first acceptance process is a process of accepting selection of a calling process of calling the second person Pb, on the basis of a first operation by the first person Pa for the contact sensor 11. The second acceptance process is a process of accepting selection of execution of a calling process which has been selected in the first acceptance process, on the basis of a second operation by the first person Pa for the contact sensor 11. The third acceptance process is an acceptance process of accepting selection of a calling reason for calling the second person Pb, on the basis of a third operation by the first person Pa for the contact sensor 11.

The acceptance processes S11 can be repeated until acceptance of the (selection of) “execution” of the calling process (that is, the second acceptance process) (the step S12). For example, either of the first acceptance process and the third acceptance process may come before the other, provided that they precede the second acceptance process. Further, the first acceptance process and the third acceptance process may be performed simultaneously. That is, the first acceptance process can serve also as the third acceptance process. For example, in a case where, as described later, the option to be selected in the first acceptance process indicates both a “calling process” and a “calling reason”, selection of this option corresponds to both of the first operation and the third operation (the first acceptance process and the third acceptance process).

After the second acceptance process (YES in the step S12), the control section 14 executes a calling process. The control section 14 controls the second information provision section 12b to cause the second information provision section 12b to provide the second information for calling the second person Pb with use of, for example, image information, sound information, or both of them. This second information preferably include information on a calling reason selected in the third acceptance process. The second person Pb called moves from the region Rb to the region Ra and, for example, checks and handles a condition of the first person Pa.

The following will discuss examples in which the calling system 1 is applied to (i) a medical/nursing care and (ii) a work site. For simplicity, it is assumed that the first information provision section 12a and the second information provision section 12b are display devices for providing, as the information, image information.

The following will describe an example in which the calling system 1 is applied to a medical/nursing care. It is assumed that the first person Pa is a subject of a medical or nursing care and, for example, is lying on a bed.

FIG. 3 is a view schematically illustrating a relationship between the first information provided by the first information provision section 12a and an operation of the contact sensor 11. The first information provision section 12a is displaying “calling”, “bed adjustment”, “confirmed”, and “return”, as options. The first information provision section 12a displays, as a cursor AR, a point on the contact surface 11a with which a finger F of the first person Pa is in contact.

The “calling” is a first option for selecting the calling process. The “bed adjustment” is an option for selecting a calling process for adjustment of the bed. The “bed adjustment” is the first option for selecting the “calling process” and, at the same time, is a third option for selecting the “calling reason”. The “confirmed” is an option for confirming the option selected. While the “calling” or the “bed adjustment” is selected, the “confirmed” functions as a second option for selecting execution of the calling process. The “return” is an option for displaying other options which are currently not displayed on the first information provision section 12a.

The first person Pa selects the “calling” or the “bed adjustment” and then can select the “confirmed” for the calling. The control section 14 accepts the selection and executes the calling process. That is, the control section 14 controls the second information provision section 12b to cause the second information provision section 12b to provide the second information for calling the second person Pb. In this case, when the “bed adjustment” has been selected, the second information includes information on the “bed adjustment”.

After the first person Pa has selected the “calling”, the first person Pa can select an option for a reason for the calling. Information on options for the calling reason is provided by the first information provision section 12a. For example, setting as follows enables the “confirmed” to be used as an option for providing information on options for the calling reason. That is, the first information provision section 12a is set to, in a case where the “calling” is selected, provide the information on the options for the calling reason in response to the selection of the “confirmed”, instead of executing the calling process. Alternatively, a configuration is possible in which the first information provision section 12a is caused to display, together with the “calling” and the like, options only for the “calling reason” for providing information on the options for the calling reason.

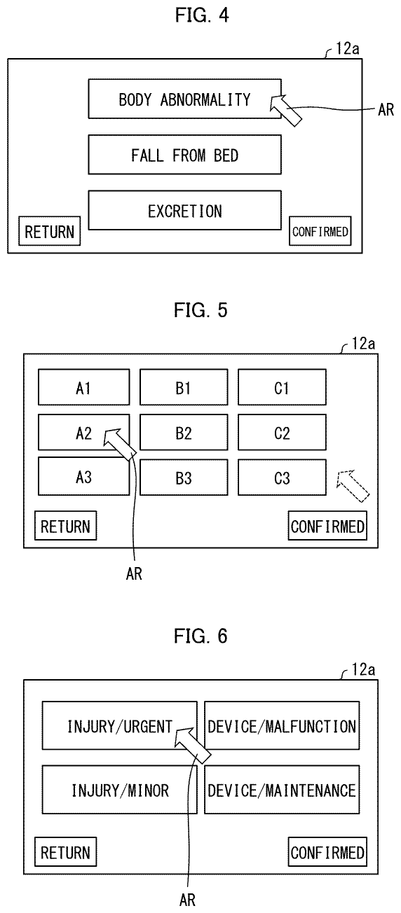

FIG. 4 is a view schematically illustrating an example of information on options for the calling reason which is provided by the first information provision section 12a. The first information provision section 12a is displaying, as the options, “body abnormality”, “fall from bed”, “excretion”, “confirmed”, and “return”.

The “body abnormality”, “fall from bed”, and “excretion” are each a third option for selecting a calling reason. The “confirmed” is an option for confirming the option selected. The “return” is an option for displaying other options which are currently not displayed on the first information provision section 12a.

After the first person Pa has selected the third option for selecting a calling reason, the first person Pa can select the second option for selecting execution of the calling process. The control section 14 accepts the selection of the third option and then executes the calling process. In the calling process, the second information provided by the second information provision section 12b includes information on the calling reason (either one of the “body abnormality”, “falling from bed”, and “excretion”) which has been selected in the third acceptance process.

The following will describe an example in which the calling system 1 is applied to a work site. It is assumed that the first person Pa is an operator and the second person Pb is a supervisor of the operation.

It is assumed that the work site is divided into regions A1 to A3, B1 to B3, and C1 to C3 in which respective operators are working. That is, a plurality of first persons Pa (operators) can call the second person Pb. In order to match the number of the plurality of first persons Pa, a plurality of contact sensors 11 and a plurality of first information provision sections 12a are provided. Each of the first persons Pa wears the garment C having the contact sensor 11 and can be provided with the information from the corresponding one of the first information provision sections 12a.

FIG. 5 is a view schematically illustrating an example of the information on the options provided by the first information provision section 12a. The first information provision section 12a is displaying, as the options, the “A1” to “A3”, the “B1” to “B3”, the “C1” to “C3”, the “confirmed”, and the “return”.

The “A1” to “C3” are each a first option for selecting the calling process and each serve also as an option for specifying a calling place (from among regions A1 to C3). That is, selection of “A1” means selection of calling the second person Pb to the region A1. When, as described above, the options for the calling process each serve also as option for information in connection with the calling process (here, a place to which the second person Pb is to be called), it becomes easy for the first person Pa to promptly call the second person Pb. In addition, it is possible to standardize the first information in the different regions A1 to C3.

The “confirmed” is an option for confirming the option selected. While either one of the “A1” to “C3” is selected, the “confirmed” serves as the second option for selecting execution of the calling process. The “return” is an option for displaying other options which are currently not displayed on the first information provision section 12a.

The first person Pa selects either one of the “A1” to “C3” on the basis of the contents displayed on the first information provision section 12a and then can select the “confirmed” for calling. The control section 14 accepts the selection and executes the calling process. That is, the control section 14 controls the second information provision section 12b to cause the second information provision section 12b to provide the second information for calling the second person Pb. In this case, when the “A1” has been selected, the second information includes information indicating that a place from which the call comes is the region “A1”.

For example, after the first person Pa has selected the “A1”, the first person Pa can select an option for a calling reason. Information on options for the calling reason is provided by the first information provision 12a. example, section For the first information provision section 12a is caused to display, together with the “A1”, an option only for the “calling reason” for providing information on the options for a calling reason and provide information on options for a calling reason in response to the selection of the “calling reason”. Alternatively, after the selection of “A1”, the first information provision section 12a may be caused to provide information on the options for a calling reason in response to the selection of the “confirmed”, instead of executing the calling process. In this case, the “confirmed” illustrated in FIG. 5 does not function as the second option for selecting execution of the calling process.

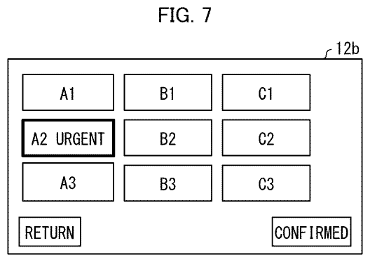

FIG. 6 is a view schematically illustrating an example of information on the options for the calling reason which is provided by the first information provision section 12a. The first information provision section 12a displays, as options, “injury/urgent”, “injury/minor”, “device/malfunction”, “device/maintenance”, “confirmed”, and “return”.

The “injury/urgent” indicates that an operator is injured and requires an urgent t treatment. The “injury/minor” indicates that an operator is injured but the injury is relatively minor. The “device/malfunction” indicates that there is a malfunction in the device. The “device/maintenance” indicates that the device requires maintenance. The “confirmed” is an option for confirming the option selected. The “return” is an option for displaying other options which are currently not displayed on the first information provision section 12a.

After the first person Pa has selected the third option for selecting a calling reason, the first person Pa can select the second option for selecting execution of the calling process. The control section 14 accepts the selection of the third option and then executes the calling process. In the calling process, the second information provided by the second information provision section 12b includes information on the calling reason (either one of the “injury/urgent”, “injury/minor”, “device/malfunction”, and “device/maintenance”) which has been selected in the third acceptance process.

FIG. 7 is a view schematically illustrating an example of the second information provided by the second information provision section 12b. Here, in response to selection of the “injury/urgent”, the second information provision section 12b displays, as the “A2 URGENT”, emergence of an injured person of urgency in the region A2.

Here, the second information provision section 12b displays options similar to those on the image displayed on the first information provision section 12a in FIG. 5, emphasizes the selected option in which the calling process has been executed, and also displays the calling reason. That is, the second information provision section 12b is used for both monitoring of the operation condition of the contact sensor 11 and the calling process. When, as described above, monitoring of the operation condition of the contact sensor 11 is possible through the second information provision section 12b, it is easy for the second person Pb to be informed of the condition of the first person Pa before the calling process.

As such, the calling system 1 allows the first person Pa to operate the contact sensor 11 on the basis of assistance of the first information provision section 12a to call the second person Pb. The first person Pa can select a calling reason to present the calling reason at the calling.

### [Recap]

A calling system in accordance with Aspect 1 is a calling system that allows a first person to call a second person, the calling system including: a mutual capacitive contact sensor which has fibrous electric conductors and which is to be operated by the first person, the mutual capacitive contact sensor being formable integrally with a garment to be worn by the first person or being attachable to the garment; a first information provision section configured to provide the first person with first information for assisting the first person in operating the mutual capacitive contact sensor; a second information provision section configured to provide the second person with second information for calling the second person; and a control section configured to cause the second information provision section to provide the second information in accordance with an operation by the first person for the mutual capacitive contact sensor, so as to execute a calling process of calling the second person.

The calling system 1 in accordance with Aspect 1 enables the first person Pa to operate the contact sensor 11 on the basis of assistance of the first information provision section 12a to call the second person Pb.

A calling system in accordance with Aspect 2 is configured, in the calling system in accordance with Aspect 1, such that the control section executes the calling process after the control section has executed: a first acceptance process of accepting selection of the calling process in accordance with a first operation by the first person for the mutual capacitive contact sensor; and a second acceptance process of, in accordance with a second operation by the first person for the mutual capacitive contact sensor, accepting selection of execution of the calling process which has selected in the first acceptance process.

The calling system 1 in accordance with Aspect 2 enables the first person Pa to select a calling process and then select execution of the calling process selected. This leads to reduction in occurrence of incorrect calling process.

A calling system in accordance with Aspect 3 is configured, in the calling system in accordance with Aspect 2, such that the mutual capacitive contact sensor has a contact surface with which the first person is able to make a direct or indirect contact; the calling system includes a storage section that stores a table indicative of a correspondence between a plurality of positions on the contact surface and a plurality of options; the plurality of options include a first option for selecting the calling process and a second option for selecting execution of the calling process; and the control section executes the first acceptance process and the second acceptance process in accordance with a position on the contact surface with which position the first person has made contact and the table.

The calling system 1 in accordance with Aspect 3 enables the control section 14 to accept selection of the first option for selecting a calling process and selection of the second option for selecting execution of the calling process on the basis of the table T indicative of a correspondence between the plurality of positions on the contact surface 11a of the contact sensor 11 and the plurality of options.

A calling system in accordance with Aspect 4 is configured, in the calling system in accordance with Aspect 2 or 3, such that after the control section has executed a third acceptance process of, in accordance with a third operation by the first person for the mutual capacitive contact sensor, accepting selection of a calling reason for calling the second person, the control section executes the calling process; and the second information provided in the calling process includes information on the calling reason which has been selected in the third acceptance process.

The calling system 1 in accordance with Aspect 4 enables the first person Pa to select the calling reason for calling the second person Pb to present the calling reason selected at the calling of the second person Pb.

A calling system in accordance with Aspect 5 is configured, in the calling system in accordance with any one of Aspects 1 to 4, such that the mutual capacitive contact sensor is attached to or is formed integrally with at least one selected from the group consisting of a left sleeve and a right sleeve of the garment.

The calling system 1 in accordance with Aspect 5 enables the first person Pa to, with a dominant hand, operate the contact sensor 11 disposed at a sleeve opposite to the dominant hand. The first person Pa wearing a garment in which the contact sensor 11 is disposed at a sleeve opposite to a dominant hand can easily operate the contact sensor 11 with the dominant hand. Use of the garment C in which the contact sensors 11 are disposed at both the left sleeve and the right sleeve enables the first person Pa to easily operate the contact sensor 11 with a dominant hand in either case where the dominant hand of the first person Pa is right or left.

The present invention is not limited to the embodiments, but can be altered by a skilled person in the art within the scope of the claims. The present invention also encompasses, in its technical scope, any embodiment derived by combining technical means disclosed in differing embodiments.

1. A calling system that allows a first person to call a second person, the calling system comprising:
a mutual capacitive contact sensor which has fibrous electric conductors and which is to be operated by the first person, the mutual capacitive contact sensor being formable integrally with a garment to be worn by the first person or being attachable to the garment;
a first information provision section configured to provide the first person with first information for assisting the first person in operating the mutual capacitive contact sensor;
a second information provision section configured to provide the second person with second information for calling the second person; and
a control section configured to cause the second information provision section to provide the second information in accordance with an operation by the first person for the mutual capacitive contact sensor, so as to execute a calling process of calling the second person.

2. The calling system according to claim 1, wherein the control section executes the calling process after the control section has executed:
a first acceptance process of accepting selection of the calling process in accordance with a first operation by the first person for the mutual capacitive contact sensor; and
a second acceptance process of, in accordance with a second operation by the first person for the mutual capacitive contact sensor, accepting selection of execution of the calling process which has selected in the first acceptance process.

3. The calling system according to claim 2, wherein:
the mutual capacitive contact sensor has a contact surface with which the first person is able to make a direct or indirect contact;
the calling system includes a storage section that stores a table indicative of a correspondence between a plurality of positions on the contact surface and a plurality of options;
the plurality of options include a first option for selecting the calling process and a second option for selecting execution of the calling process; and
the control section executes the first acceptance process and the second acceptance process in accordance with a position on the contact surface with which position the first person has made contact and the table.

4. The calling system according to claim 2, wherein:
after the control section has executed a third acceptance process of, in accordance with a third operation by the first person for the mutual capacitive contact sensor, accepting selection of a calling reason for calling the second person, the control section executes the calling process; and
the second information provided in the calling process includes information on the calling reason which has been selected in the third acceptance process.

5. The calling system according to claim 1, wherein the mutual capacitive contact sensor is attached to or is formed integrally with at least one selected from the group consisting of a left sleeve and a right sleeve of the garment.
